(19) United States
(12) Patent Application Publication
(10) Pub. No.: US 2021/0225477 A1
(43) Pub. Date: Jul. 22, 2021

(54) METHOD AND APPARATUS FOR ASSISTING DRUG DELIVERY

(71) Applicant: CAMBRIDGE MEDTECH SOLUTIONS LTD, Stoke-On-Trent ST4 2QY (GB)

(72) Inventors: Stuart Brian William KAY, Stoke-On-Trent (GB); Sebastien Antoine Yves CUVELIER, Stoke-On-Trent (GB)

(21) Appl. No.: 16/314,299

(22) PCT Filed: Jun. 29, 2017
(86) PCT No.: PCT/GB2017/051905

§ 371 Date: Dec. 28, 2018

(30) Foreign Application Priority Data
Jun. 29, 2016 (GB) 1611324.3

(51) Int. Cl. G16H 20/13; A61M 15/00

(52) U.S. Cl. CPC G16H 20/13; A61M 2016/0018; A61M 2205/3375; A61M 15/00

(57) ABSTRACT

A method, apparatus, computer program, programmable device and system are all disclosed for the detection of the actuation of an inhaler and breath of a user from audio data. The method comprises identifying, based on a high frequency band of the audio data, actuation of the inhaler. The method additionally comprises identifying, based on a low frequency band of the audio data and based on the identified actuation of the inhaler, an interval of the audio data comprising the breath of the user.

### FIELD

The present invention relates to methods and apparatus for assisting operation of drug delivery devices such as inhalers, and more particularly to methods and apparatus for providing guidance to a user of such a device based on the processing of audio signals to identify the relative timing of the actuation of an inhaler and the breath of a user.

### BACKGROUND

A large amount of medication is wasted because patients are not aware how to take it properly. One example of such waste is through the misuse of inhalers. Inhalers such as metered-dose inhalers are devices that deliver a set amount of medication to the user via the lungs. The medicine is contained in a can that can be released in a short burst that can then be inhaled by the patient. Specialised equipment is often needed to detect such misuse in order to better educate patients.

WO 2014/033229 describes an inhaler comprising a microphone, microprocessor, battery and memory means. The inhaler comprises the microphone so that it is a set distance from the inhalation region, and can be calibrated as such. The microphone is then used to record the sound so that mel frequency cepstral coefficients can be calculated so that the inhalation and exhalation of the user can be established. This can be used to determine if the inhaler was used correctly.

WO 2015/006701 describes an inhaler with a monitor that can be affixed to the exterior of an inhaler, and that can communicate data with a device. This system requires the monitor to be situated on the inhaler to function. Sounds are recorded by the microphone and compared to pre-loaded acoustic waveforms in order to ascertain the inhalation flow rate through the inhaler and to identify events.

### SUMMARY

Aspects and examples of the invention are set out in the claims.

### BRIEF DESCRIPTION OF DRAWINGS

FIG. 1 shows a block diagram representing an apparatus comprising an inhaler and an electronic device.

FIG. 2 shows a method of identifying the actuation of the inhaler and periods of established flow associated with intervals comprising the breath of a user.

FIG. 3 shows a further method of identifying the actuation of the inhaler and periods of established flow associated with intervals comprising the breath of a user.

FIG. 4 shows yet a further method of identifying the actuation of the inhaler and periods of established flow associated with intervals comprising the breath of a user.

FIG. 5 shows a method of determining, from the identified actuation of the inhaler and breath of the user, what message to show the user regarding their use of the inhaler.

FIG. 6 shows a graph representing the high frequency band of the audio data of a first example audio signal.

FIG. 7 shows a graph representing the low frequency band of the audio data of the first example audio signal.

FIG. 8 shows a graph representing the modified low frequency band of the audio data of the first example audio signal.

FIG. 9 shows a graphical illustration of the identified actuation point and the identified high flow and established flow of the first example audio signal.

FIG. 10 shows a graph representing the high frequency band of the audio data of a second example audio signal.

FIG. 11 shows a graph representing the low frequency band of the audio data of the second example audio signal.

FIG. 12 shows a graph representing the modified low frequency band of the audio data of the second example audio signal.

FIG. 13 shows a graphical illustration of the identified actuation point and the identified established flow of the second example audio signal.

### DETAILED DESCRIPTION

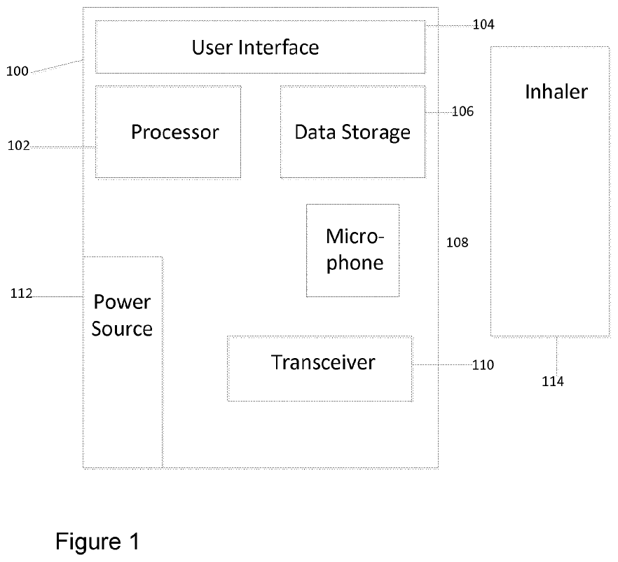

FIG. 1 shows an apparatus comprising an electronic device 100, such as a mobile phone, and an inhaler.

The electronic device 100 comprises a user interface 104, such as a touchscreen, a processor 102, data storage 106, a power source 112 and a microphone 108.

The processor 102 is connected to the data storage 106 so that it can access the data storage when needed. The user can give the handset commands through the user interface 104. The microphone 108 is used to record the sound of the inhaler 114 being actuated and of the user breathing. This generates audio data that can be stored in the data storage.

The processor 102 is configured to analyse the audio data to identify the actuation of the inhaler 114 and the breathing of the user. The processor may access the data storage to obtain instructions for performing the analysis that may be stored in the data storage.

The identification of the actuation of the inhaler, and of the breath of the user allows the processor to determine if the inhaler was used correctly. It also enables the processor to determine, if the inhaler was used incorrectly, what mistake was made by the user. The electronic device can be configured to then provide feedback to the user. The feedback could constitute advice on how to improve the use of the inhaler, information on the errors that were made by the user or both. This can increase the likelihood of the user using the inhaler correctly and consequently reduce the amount of medication that is wasted.

To do so, the processor 102 may access the data storage 106 to access a pre-set message associated with the identified actuation and breath. This may be a message saying that the inhaler 114 has been used correctly, a message associated with an error that the user has performed or both.

The electronic device 100 may also have a transceiver 110 so that it can send and receive data from another source. This may be used for passing the analysed data to a third party. This could be so that they can analyse the amount of people who are correctly administering their medicine. This is useful as it can help with the interpretation of large studies and help find a more reliable efficacy of a drug. The transceiver 110 may also allow for updates to the method so that it can be fine-tuned so that the user gets better, more effective feedback, or a better user experience.

Additionally if the phone has limited processing power, it may obtain the audio data and send it to a third party where it is analysed. For example, this may be done during a telephone call to the third party, or by recording the audio content and then sending it on. It can then receive a message from the third party with a message detailing if the inhaler was used properly, and if not how the use of the inhaler can be improved.

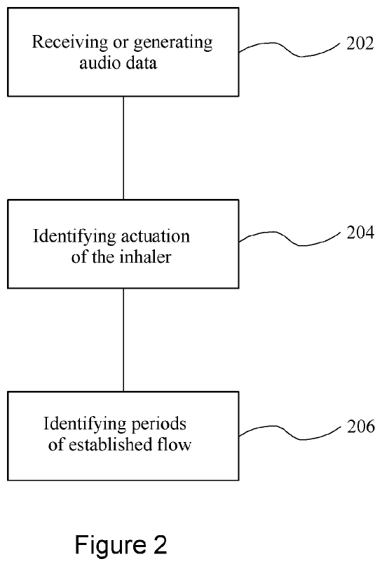

FIG. 2 shows a method for detecting the actuation of an inhaler and the breath of a user from audio data. This method can be carried out by the device shown in FIG. 1 and described in the accompanying description, and can be carried out by any other suitably configured device.

Step 202 comprises receiving or generating audio data. When the method is commenced a rolling buffer with a capacity is used. The method may be commenced when the user begins recording before the use of the inhaler. The electronic device is then triggered, emptying the buffer to generate the audio data. The electronic device may be triggered by the user confirming they have used the inhaler. The capacity of the rolling buffer may be 20 seconds. The data may be collected elsewhere and be sent to the electronic device via the transceiver. The audio data may comprise time-frequency data indicating the energy in a plurality of frequency bands as a function of time. These bands may be a high frequency band of the audio data, and a low frequency band of the audio data.

The next step is identifying the actuation of the inhaler 204. This identification is based on a high frequency band of the audio data. The processor is configured to perform this step by analysing the high frequency band of the audio data. For example, the processor may compare the high frequency band of the audio data to a threshold to determine periods of the data that exceed the threshold.

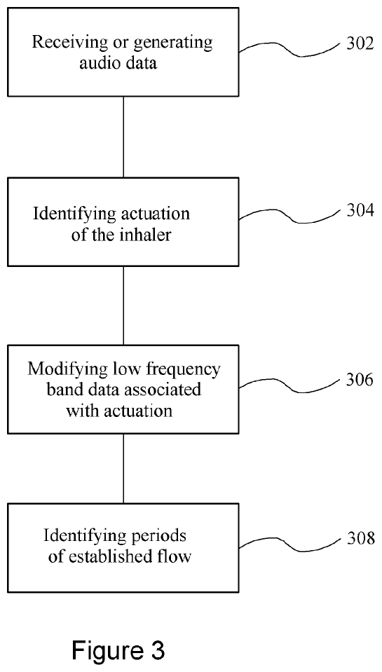

Next, an interval of the audio data comprising the breath of a user is identified 206. This identification is based on a low frequency band of the audio data and is also based on the identified actuation of the inhaler. The processor is also configured to perform this step by analysing the identified actuation of the inhaler and the low frequency band of the audio data. For example, the processor may be configured to modify its treatment of low frequency band audio data associated with the identified actuation. This may be done by excluding that data, replacing it with substitute data, or weighting it in some way. One example of such an implementation is disclosed below with reference to FIG. 3.

The method may include the process of frequency transforming time domain audio data to obtain the different frequency bands. In some cases however the time domain audio data may have already been transformed before the method is begun so that the energy of each frequency band as a function of time has already been determined. Any frequency transformation may be used. Examples include a Fourier transform, a cosine transform and a wavelet transform. Each of these may be a fast, or discrete version of each transform, such as a fast Fourier transform. To do so the audio data may be stored in the data storage. The processor may then retrieve it and perform the transformation. The result may be stored in the data storage before further analysis. One way that a frequency transform could be performed on the time domain audio data may be to split the time domain audio data into a series of temporal chunks (the chunks may overlap with one another). A frequency transform may then be calculated for each temporal chunk. The transforms of the chunks produce a series of transforms indicating how the energy in each frequency band evolves during the time interval associated with the corresponding chunk. Other transformation techniques may also be used in order to determine this evolution. Continuous wavelet transforms for example do not require the subdivision of data in this way. Sliding window approached may be used.

By performing this method the actuation of the inhaler and the breath of the user can be found from the audio data. This can then be used to find if the user has used the inhaler in the correct manner, and can be used to inform them about how to do so effectively. This method accomplishes this without the need for specialist hardware, as it can be implemented on devices (such as telecommunications handsets) already owned by the user or by a carer, family member, health care professional or friend. The method also does not require the audio data to be collected from a specified position, or a set distance from the point of inhalation. This flexibility allows the method to be performed in an uncontrolled environment and without specialist equipment. This makes it easier for a user to check if they are administering their medication correctly, and therefore will cut down the amount of medication that is wasted through misuse.

FIG. 3 shows an example of a method for detecting the actuation of an inhaler and the breath of a user from audio data such as that described with reference to FIG. 2. This method can be carried out by the device shown in FIG. 1 and described in the accompanying description, and can be carried out by any other suitably configured device.

In addition to the features of the method described with reference to FIG. 2, the method of FIG. 3 further involves the modification of the low frequency band data associated with the actuation of the inhaler 306. As the actuation of the inhaler has been identified in the high frequency band of the audio data, by cross referencing the peaks in the low frequency band for peaks that are associated with the actuation of the inhaler, these peaks can be ignored for the purpose of further analysis. The processor can perform this modification by accessing the low frequency band of the audio data and the data associated with the identified actuation of the inhaler from the data storage, and then performing the modification.

There are several ways in which this can be accomplished. For example, the data associated with the actuation of the inhaler can be replaced with substitute data that is not associated with the actuation of the inhaler. One way of doing this would be to identify preceding data (the level before the associated data) and subsequent data (after the associated data) and to replace the data in between. This data could be replaced with a continuous function. One such function could be a straight line between the two points, alternatively quadratic or other equations could be used to fit between the two points. Alternatively a weighting function could be used either to scale the low frequency band of the audio data during the interval of actuation, or to adjust the fit of a data model (e.g. using regression) to the low frequency band of the audio data so that contributions from the actuation can be modelled out. This could be achieved by giving no weighting, or little weighting to periods associated with the actuation of the inhaler when analysing the low frequency band, so that periods of established flow and intervals comprising the breath of a user can be identified accurately. Other examples of possible modification techniques include overlaying the two frequency bands, or subtracting the data from one another. Any suitable technique can be used to modify the data in the low frequency band so that the breaths of the user can be accurately identified. The instructions on how to perform the weighting could be stored in the data storage, or could be encoded in the processor itself. After accessing the instructions the processor can then perform them and return the results to the data storage.

The identifying of the periods of established flow 308, or the intervals comprising the breath of the user may then be based on the modified low frequency band of the audio data. This is still therefore based on the low frequency and on the identified actuation of the inhaler as these contribute to the modified low frequency data. This can be performed by the processor using the results of the modification, the processor may have to access this, or the low frequency band of the audio data from the data storage.

The use of the modification allows the identified actuations to not be re-identified as breaths of a user. This makes the results more accurate, and means that the method can accurately assess if the user has used their medication in the correct manner.

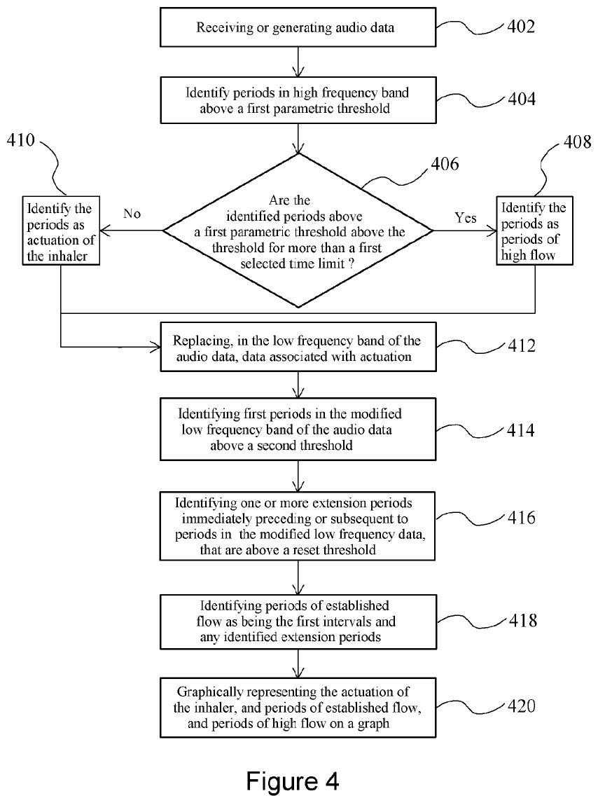

FIG. 4 shows yet a further refined method for detecting the actuation of an inhaler and the breath of a user from audio data based on the methods shown in FIGS. 2 and 3 and described in the accompanying description. This method can be carried out by the device shown in FIG. 1 and described in the accompanying description, and can be carried out by any other suitably configured device.

Firstly, as above, the audio data must be recorded, generated or received 402. At this point a spectrogram may be formed of the audio data. The recording may be performed by the microphone 108, or the generation may be performed by the processor 102, alternatively the audio data may be received via the transceiver 110. If the audio data is transformed to form a spectrogram, then the processor can perform this task.

The next step is to identify periods in the high frequency band of the audio data above a first parametric threshold 404. The parametric threshold may be based on the maximum height of the high frequency band data, or it may be a set constant threshold. The processor may access the high frequency band of the audio data, possibly from the data storage, it will then determine the maximum value in this data set, and from this calculate the first parametric threshold, and then determine periods in the high frequency band that are above the high frequency threshold. If the threshold is a pre-set value this may be stored in the data storage and accessed by the processor.

The periods that are identified are then compared to a first selected time limit 406. If the identified period is above the first parametric threshold for longer than the duration of the selected time limit then the period is identified a period of high flow 408. The first set time limit may be 0.4 seconds.

If the identified period is above the first parametric threshold for less time than the duration of the selected time limit then the period is identified as being an actuation of the inhaler 410. The first selected time limit may be stored in the data storage and accessed by the processor. The processor may then compare the identified periods above the first parametric threshold with the first selected time limit to determine if the identified period is an actuation point, or a period of high flow.

The next step, in this example, is to replace, in the low frequency band of the audio data, data that is associated with the actuation of the inhaler 412. This can be done by identifying peaks in the low frequency band that correspond to peaks in the high frequency band that have been identified as being associated with the actuation of the inhaler. The data in the low frequency band that is associated with actuation can then be replaced. This can be by substitution, or by the use of a weighting function. As discussed in relation FIG. 2, the processor may be configured to perform the method step, or it may access the instructions on how to do this from the data storage. Then the processor may modify the low frequency band of the audio data.

After this the modified low frequency data can be analysed. The first step of doing this is to identify periods in the modified low frequency band of the audio data above a second parametric threshold 414. This threshold may be based on the maximum value of the modified low frequency band data, or it may be a set value. Periods above the second parametric threshold are first periods. The processor may calculate the second parametric threshold by determining the maximum value of the low frequency band (this may be done based on the modified low frequency band data), and then determine the threshold based upon this. Then the processor can compare the data with the threshold to find periods where the low frequency data is above the threshold.

Extension periods are then identified 416. These are periods immediately preceding or subsequent to the first periods that are above a reset threshold. The reset threshold may also be based on the maximum value of the modified low frequency band data, or it may be a set value. There may not be any extension periods, or there may be up to two per first period. The reset threshold may be calculated in the same way, or it may be stored in the data storage and accessed by the processor. The processor may then identify any extension periods. Additionally, extension periods may be identified for periods of identified high flow in the high frequency band of the audio data in the same manner.

The next step is to identify an interval comprising breath of a user, or periods of established flow 418. These are the first periods and their associated extension periods combined. This is the breathing that the user should aim for when administering their medication from their inhaler. The processor can identify these periods form the determined first periods and established flow periods.

Finally, the last step comprises graphically representing the actuation of the inhaler, the periods of established flow and the periods of high flow on a graph 420. This enables the user, a pharmaceutical company or data analyst to see if the inhaler has been administered correctly, or if there is room for improvement. A message may be relayed to the user at this point to help encourage the correct use of the inhaler. The processor may form a graphical representation of the inhaler, and may access a template of the illustration from the data storage to do so. The processor can then send this to the user interface to display the illustration to the user.

By graphically illustrating the results, especially on a device that the user may have to hand at most times, the user can easily keep track of their inhaler use, and any errors that they have made. Children now commonly have phones, and may be some of the users of inhalers most prone to errors. This continual reinforcement may be an especially powerful tool in teaching use of an inhaler properly. This is very important as inhalers can be used in serious situations, such as during an asthma attack, when the use of it may have direct consequences on the wellbeing of the user.

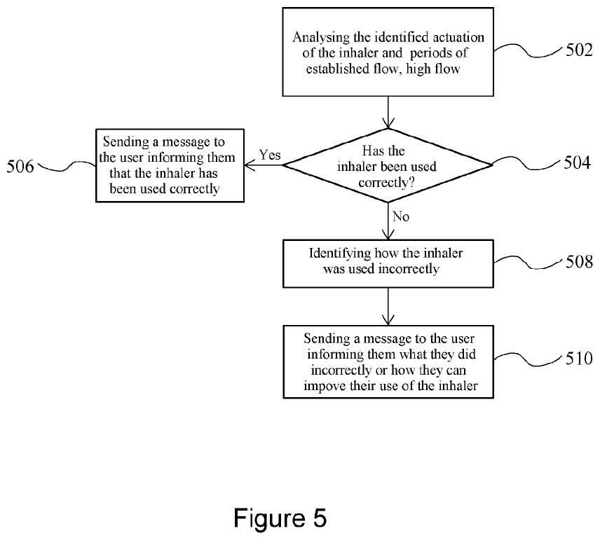

FIG. 5 shows a method for determining what message to send to a user based on their use of the inhaler. This method can be carried out by the device shown in FIG. 1 and described in the accompanying description, and can be carried out by any other suitably configured device.

The first step is to complete the analysis of the audio data, so that the actuation, periods of high flow and periods of established flow are identified 502. This can be graphically illustrated, alternatively however it may not be and the data can simply be used. This can be done using the methods shown in FIGS. 2-4 and the accompanying description.

The next step is from this analysis to find whether the inhaler has been used correctly, or incorrectly 504. If the inhaler has been used correctly, as shown in example 2, then a message can be sent to the user informing them that the inhaler was used correctly 506. This may also have positive reinforcement (such as a message saying Good Job! or perhaps, if the user is a child, earning points) so that the user continues to use their inhaler correctly. The processor may compare the results of the identification of the actuation and the user breath with a template of an ideal result stored in the data storage. The processor can then determine whether the user used the inhaler correctly.

If however the user has not used their inhaler correctly it is important to identify how they have used it incorrectly and what error has occurred 508. The processor can then determine this in a similar way to above. This time comparing the result to a set of templates showing a plurality of different errors, and possibly combinations of errors to determine how the user has used the inhaler incorrectly. This step and the step above may be combined into one step.

A message can then be sent informing the user what they did wrong, or alternatively (or additionally) how they may improve their use of the inhaler 510. The processor can determine which of the errors has occurred, and then can access the corresponding message relating to this error that may be stored in the data storage.

This method allows a message to be selected from first message data, to be sent to the user. The selection is based on the relative timing of the actuation and the interval.

The message data may be partially predefined and comprise a first message indicating the correct operation of the inhaler, and a second message comprising training instructions.

The message may be selected based on the actuation being in the interval comprising the breath of the user. This may indicate a successful use of the inhaler. Alternatively the message may be based on the actuation not being in the interval. This may indicate that the inhaler was used incorrectly.

Some examples of errors that may occur during use include, but are not limited to: actuating the inhaler more than once, not actuating the inhaler, forgetting to breath, breathing in too quickly, coughing, sneezing, the background noise being too loud to accurately identify the actuation and breath of the user, not waiting a set period of time after inhaling the medicine before exhaling, not actuating the inhaler at the beginning of a breath, actuating the inhaler without breathing and actuating the inhaler during a period of high flow.

Each of these, and any other possible errors may be associated with a phrase or message that can be shown to the user. These may simply be factual to tell the user what has gone wrong, or may include tips and encouragement. Multiple errors can be identified and a message with several errors in can be shown to the user.

It may be possible to simply record or generate the audio data and send this to a central server. The central server may then perform the analysis and only send back the feedback message to the user. This may be useful if the user does not possess a smartphone. This could be very useful in parts of the third world where smartphone usage is low, but where it is important that medication reaches people and that it is used correctly as it is a scarce resource.

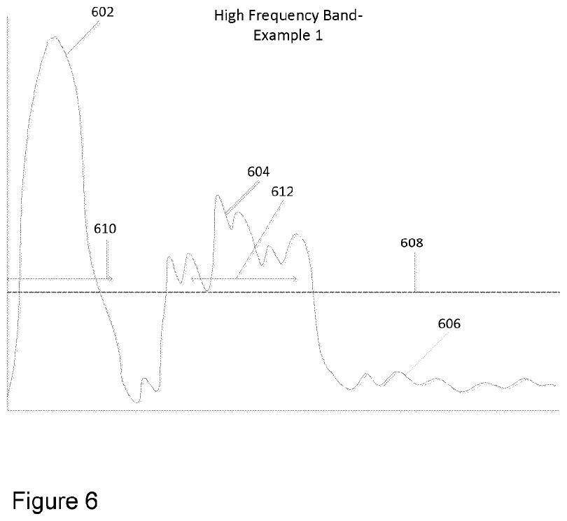

FIG. 6 is a graph showing the signal in the high frequency band of the audio data for a first audio recording (referred to herein as example 1).

This signal shows a clear sharp point 602 followed by a broader lower peak 604 and then noise 606. The dashed line across the graph represents the first parametric threshold 608. In this example the first parametric threshold is set at 30% of the maximum value recorded in the signal. This threshold is crossed in two distinct sections.

The first section 602 comprises a sharp narrow peak that rises to the maximum value recorded in the signal. This peak occurs for a short amount of time. The first arrow 610 shows a first selected time limit. The sharp narrow peak is above the first parametric threshold for less time than the first selected time limit. Therefore the sharp narrow peak is associated with an actuation of the inhaler.

The second section 604 is broader and lower. It rises to a peak value and then is relatively constant (there is of course some deviation) for a period of time. The second arrow 612 also shows the first selected time limit. The broad peak is above the first parametric threshold for more time than the first selected time limit 612. Therefore the broad peak is not associated with the actuation of the inhaler. Instead the broad peak is associated with a period of high flow.

The first parametric threshold 608 may not be based on the height of the maximum point in the signal, or it may not be based solely on that point. For example it could be a pre-set constant threshold, or it could be calculated from the average of the highest two peaks (if there are two peaks), or in another way. The purpose of the threshold is so that only genuine signals are considered for the actuation of the inhaler, or as periods of high flow. Any threshold that fulfils those criteria could be used. There may be an additional second threshold (which may be a pre-set constant) calibrated so that signals below the second threshold get discarded.

The high flow may be indicative of a sharp intake of breath that is not optimal for the intake of the drugs administered from the inhaler. Alternatively it may correspond with a whistling sound caused by a spacer not being connected to the inhaler properly, or with the user coughing. It could also be caused by environmental factors. Identifying high flow can therefore indicate the incorrect use of the inhaler, or that the user needs to stand in a quieter environment in order to test their use accurately.

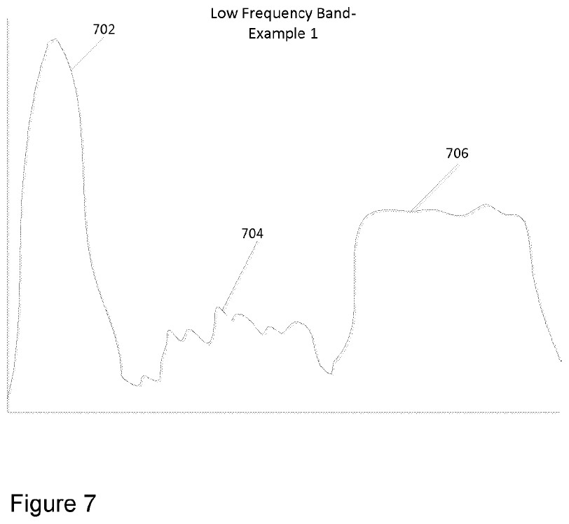

FIG. 7 is a graph showing the signal in the low frequency band of the audio data for example 1.

This signal is comprised of three distinct sections. The first section once again shows a sharp narrow peak 702. This is flowed by a section comprising a low broad peak 704. This is followed by a section that is broad, and with a relatively high peak 706.

The first section 702 appears at the same time as the identified actuation does in the high frequency band of the audio data. It is also similarly shaped, and therefore can be identified as also being caused by the actuation of the inhaler. Actuation of the inhaler can cause a broadband signal that registers in both the high frequency band of the audio signal and the low frequency band of the audio signal. One of these signals may be stronger, but in this case they are approximately equal.

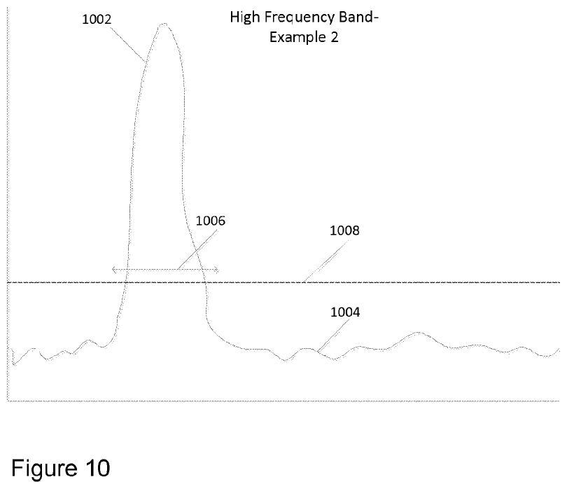

The second section 704 is broad, and relatively low. This section appears at the same time as the high flow identified in FIG. 10. It seems likely that this signal is due to the identified high flow. The signal of the high flow in the low frequency band may have a lower peak than in the high frequency band of the audio signal because it may not be present in the same range of frequencies as the actuation signal. It may disappear altogether in the low frequency band of the audio data, however high flow recorded from some sources (potentially such as coughing) may be detected approximately equally in both bands.

The third section 706 is also broad, but has a height somewhat higher than the second section. There is no corresponding peak in FIG. 10 to this peak. It is therefore likely that the source of the peak only produces low frequency signals.

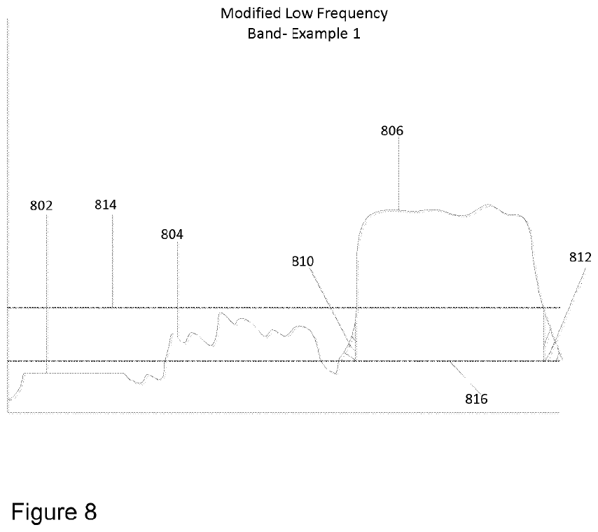

FIG. 8 is a graph that may be used in one example, showing the signal in the low frequency band of the audio signal of example 1 that has been modified.

The most striking difference between FIG. 7 and FIG. 8 is that the sharp narrow peak 702 corresponding to the first section is missing from FIG. 8. FIG. 6 was used to identify this peak as being associated with the actuation of an inhaler. Therefore when analysing the low frequency band of the audio data this data has to be ignored when attempting to identify periods of established flow associated with the breath of a user (as it has already been associated with the actuation). One way to do so is to modify the low frequency band of the audio data as shown in FIG. 8. Here a linear line 802 has been drawn across from a point immediately before the peak to a point immediately after the peak. This removes the section associated with the actuation from the low frequency band dataset. The line comprises substitute data that is used to replace data associated with the identified actuation.

Two dotted lines 814 and 816 are shown on the graph. These represent a second parametric threshold 814 and a reset threshold 816. In this example the second parametric threshold 814 is set at 50% of the maximum value of the modified low frequency band. The reset threshold 816 is set at 20% of the modified low frequency band. A number of first periods are identified as being above the second parametric threshold 814. In this example only the peak of the third section 806 is above the second parametric threshold. This is the first period. An extension period before 810, and an extension period after 812, the first period, are found. The extension periods are the areas surrounding the first period that are below the second parametric threshold, but are above the reset threshold. The first period and the extension periods must be continuous in time. This corresponds to sections 810 and 812. The combination of the first period and the extension periods comprises the breath of a user. This corresponds to a period of established flow.

The second section 804 is above the reset threshold 816, but below the second parametric threshold 814, therefore this is not identified as being associated with the breath of a user, and therefore is not established flow.

The second parametric threshold 814 and reset threshold 816 may instead be pre-set values, rather than being based on the maximum value of the modified low frequency band of the audio data. One of them may be a constant and value, and one may be calculated based on the maximum, or another suitable method for calculating the thresholds may be used. It may be possible to identify if established flow is either an inhalation or an exhalation of air. In this case another parameter may be required for analysis.

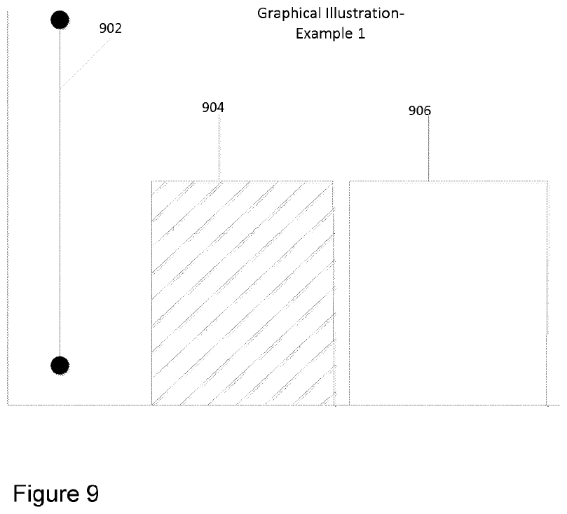

FIG. 9 shows a graphical illustration of the result of the analysis carried out on the FIGS. 6-8. Three distinct objects are shown. The first corresponds to the identified actuation point 902. The second corresponds to the identified period of high flow 904, and the third object corresponds with the breaths of the user 906, and is a period of established flow.

This shows that the actuation 902 does not take place in a period in which the user is identified as breathing 906. The user first presses the inhaler to release a dose of medication, there is then a period of high flow, and then the user breath (which may include instances of multiple breaths and multiple instances of high flow) This is not how the user should be using the inhaler, therefore when this illustration is displayed it may be accompanied, or followed, with advice on how to improve, or information on how the user is using the inhaler wrong.

In this case the advice may be to say that the user should begin to breathe deeply, and then actuate the inhaler, and then continue to breathe deeply. They should then hold their breath for a period of time before exhaling. Alternatively the advice may say that the high flow may indicate that the background noise was too loud, and that they should move to a quieter area.

The users can then look at the advice and become aware of what they are doing wrong, or how they can improve. This allows the users to take their medication more effectively in the future and reduce the amount of wasted medication, and improve the clinical results.

The data shown in the graphical illustration may be useful for pharmaceutical companies that are conducting clinical trials. In such a trial it would be advantageous to work out which proportion of the participants are taking the medication as they are supposed to, and how many are not. If a drug seems less effective, this may be because the users have not taken it effectively. This data may also identify the individual participants so that if the data from those participants not taking the medication properly was discarded the data could be re-analysed to find out the efficacy of the drug being tested. Therefore a method of sending this data, and possibly compressing the data for it to be sent, to a central server may be advantageous. This data may also be useful for large health bodies when assessing which medication they should supply to people, and which they should discontinue.

FIG. 10 is a graph showing the signal in the high frequency band of the audio data for a second audio recording (referred to herein as example 2).

There is one clear peak 1002. This peak is tall and narrow and represents the highest point in the signal in the high frequency band of the audio data. The dotted line across the graph is the first parametric threshold 1008. This is set at 30% of the maximum point in the data, however it may be set in other ways. The rest of the signal appears to be noise 1004.

The first parametric threshold 1008 may alternatively to be set as a pre-set value. It may also be a different percentage of the maximum value, or it may be based on some other criteria. Another threshold may also be utilised in order to have a noise level. This may be a preset level and may be set so that even the highest peak is ignored if it is sufficiently small.

The arrow 1006 represents the first selected time limit. The peak 1002 is above the first parametric threshold for a period of time that is less than the first selected time limit 1006. Therefore the peak is associated with the actuation of an inhaler.

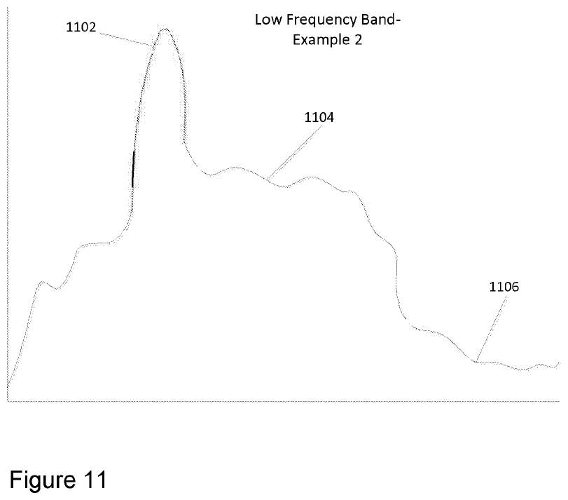

FIG. 11 is a graph showing the signal in the low frequency band of the audio data for example 2.

FIG. 11 shows a broad, high peak 1104 that contains within it an additional taller, narrow peak 1102. The narrow, sharp, tall peak is at the same time as the peak associated with actuation 1002 in FIG. 10. Therefore it is likely that this peak is also associated with the actuation of the inhaler. The rest of the signal however does not appear to be associated with the actuation of the inhaler. The period after the broad peak appears to be noise 1106.

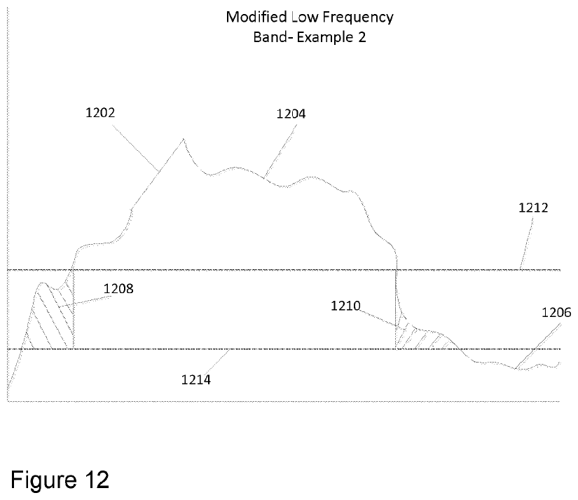

FIG. 12 is a graph that may be used in one example, showing a modified signal in the low frequency band of the audio signal of example 2.

The most striking difference between FIG. 11 and FIG. 12 is that the narrow sharp, tall peak 1102 does not appear in FIG. 12. The data associated with the peak has been substituted with replacement data. In this case a linear line 1202 has been drawn from a point before the peak, to a point after the peak. However other replacement data could have been used. For example a different continuous function could have been used to link the two points. This shows that, because the points immediately before and after the actuation peak are in a peak themselves, the substitute data conforms to this peak.

The two dashed lines 1212 and 1214 correspond to a second parametric threshold and a rest threshold respectively. The second parametric threshold 1212 is set at 50% of the maximum value of the modified low frequency band of the audio data. The reset threshold 1214 is set at 20% of the maximum value of the modified low frequency band of the audio data. The thresholds can be set in other ways.

The peak 1204 is above the second parametric threshold 1212. This peak is defined as the first period. Additionally areas immediately before and after the first period that are continuously above the reset threshold 1214 are defined as extension periods 1208, 1210. The first period combined with any associated extension periods form a period of established flow that is associated with the breath of a user. Areas below the reset threshold are not associated with the breath of the user. In this case the peak corresponds to one long breath by the user.

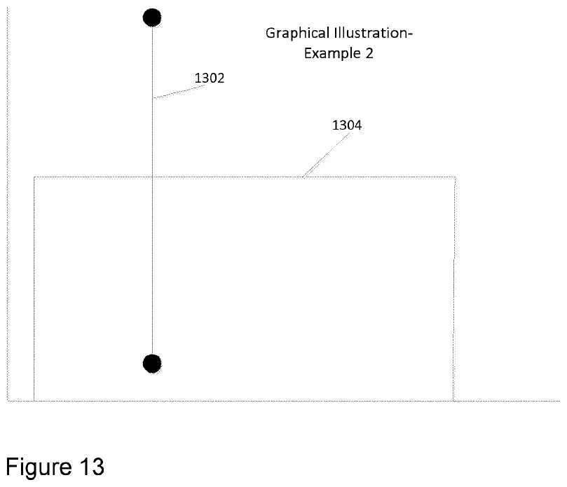

FIG. 13 shows a graphical illustration of the result of the analysis carried out on the FIGS. 10-12. Two distinct objects are shown. The first corresponds to the identified actuation point 1302. The second to the identified period of established flow associated with the breath of the user 1304.

The actuation 1302 takes place at the beginning of the identified breath 1304. The breath then continues, and is quite long. The user then does not breathe for a period of time, and no other breath is recorded.

This example shows a user correctly using the inhaler. From this section of the signal it is not possible to ascertain how long the user did not breathe after finishing their breath. Preferably the user would not breathe for five seconds after the end of the breath in which the inhaler is actuated, and more preferably the user would not breathe for ten seconds after the end of the breath.

In this instance the message would inform the user that they have correctly used the inhaler, and possibly would do so with positive reinforcement, preferably with some sort of congratulatory message. The message may also include a reminder for the user to hold their breath, preferably for 10 seconds after the end of the actuation breathe, in order to get the best results.

In an alternative embodiment rather than accessing the data storage to access instructions on how to perform the method, the processor is encoded so that it can perform the method without instruction.

In another alternative embodiment the audio data may be computer generated. A scenario may be modelled by a computer simulation, and this may provide a computer generated audio signal. This can be analysed using the same method as shown in FIGS. 2-4 and the accompanying description. This could be used for testing, or for building up a large sale database of possible measurements.

It will be appreciated from the discussion above that the embodiments shown in the Figures are merely exemplary, and include features which may be generalised, removed or replaced as described herein and as set out in the claims. With reference to the drawings in general, it will be appreciated that schematic functional block diagrams are used to indicate functionality of systems and apparatus described herein. For example the functionality provided by the data storage 100 may in whole or in part be provided by the processor 102. In addition the processing functionality may also be provided by devices which are supported by the electronic device. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein.

The processor 102 of the electronic device 100 (and any of the activities and apparatus outlined herein) may be implemented with fixed logic such as assemblies of logic gates or programmable logic such as software and/or computer program instructions executed by a processor. Other kinds of programmable logic include programmable processors, programmable digital logic (e.g., a field programmable gate array (FPGA), an erasable programmable read only memory (EPROM), an electrically erasable programmable read only memory (EEPROM)), an application specific integrated circuit, ASIC, or any other kind of digital logic, software, code, electronic instructions, flash memory, optical disks, CD-ROMs, DVD ROMs, magnetic or optical cards, other types of machine-readable mediums suitable for storing electronic instructions, or any suitable combination thereof. Such data storage media may also provide the data storage 106 of the electronic device 100.

1. A method of detecting the actuation of an inhaler and breath of a user from audio data, the method comprising:
identifying, based on a high frequency band of the audio data, actuation of the inhaler; and
identifying, based on a low frequency band of the audio data and based on the identified actuation of the inhaler, an interval of the audio data comprising the breath of the user.

2. The method of claim 1, comprising identifying a component of the low frequency audio data associated with the identified actuation and taking said component into account in the identifying of the interval of the audio data comprising the breath of the user, wherein the taking into account comprises modifying, in the low frequency band of the audio data, data associated with the identified actuation of the inhaler to provide modified low frequency data.

3-4. (canceled)

5. The method of claim 1, wherein the identifying actuation is based on comparing the high frequency band of the audio data with a first parametric threshold, wherein identifying actuation comprises identifying periods of the high frequency data which exceed the first parametric threshold for a period less than a first selected time limit, for example wherein the first selected time limit is associated with actuation.

6. (canceled)

7. The method of claim 2, wherein the modifying comprises providing substitute data to replace, in the low frequency band of the audio data, data associated with the identified actuation.

8. (canceled)

9. The method of claim 2, wherein the identifying the interval is based on comparing the modified low frequency data with a second parametric threshold, and wherein the identifying the interval further comprises identifying a first period throughout which the modified low frequency data exceeds the to second parametric threshold, and identifying at least one extension period, immediately preceding or immediately subsequent to the first period throughout which the modified low frequency data exceeds a reset threshold, wherein the first period and the at least one extension period are continuous in time.

10-13. (canceled)

14. The method of claim 1, wherein the interval comprises a period of established flow.

15. The method of claim 2, wherein the taking into account comprises applying a weighting function to the low frequency band audio data, wherein the weighting function reduces the contribution of the component in the identifying an interval.

16-18. (canceled)

19. The method of claim 1 comprising selecting, for provision to a user, a message from first message data, wherein the selecting is based on the relative timing of the actuation and the interval, wherein the first message data is at least partially predefined and comprises: a first message indicating correct operation of the inhaler; and at least one second message comprising training instructions for operation of the inhaler.

20-22. (canceled)

23. The method of claim 1 comprising identifying, in the high frequency band of the audio data, a period of high flow, having a flow rate higher than at the time of the interval, and providing an indication of the period of high flow.

24. The method of claim 23 comprising selecting for provision to a user a message from second message data, wherein the selecting is based on the timing of the period of high flow.

25. (canceled)

26. The method of claim 5 wherein the first parametric threshold is based on the high frequency band data.

27. The method of claim 1, comprising generating a graph showing the identified features of actuation and the interval of the audio data comprising the breath of the user.

28. The method of claim 1, comprising identifying one or more attributes relating to one or both of the actuation of the inhaler and breath of the user, and providing feedback to the user based on the one or more attributes, for example wherein an attribute comprises at least one of: a lack of detected actuation; lack of user breaths; a 10 second gap between intervals comprising breath of a user.

29. The method of claim 1, wherein the audio data comprises time-frequency data indicating the energy in a plurality of frequency bands as a function of time.

30. The method of claim 29 further comprising applying, to time domain audio data, a transformation to obtain said time-frequency data, for example wherein said transformation comprises one of a Fourier transform, a cosine transform, and a wavelet transform.

31. The method of claim 1, comprising the detection of coughs as broadband signals in both the high frequency band of the audio data and the low frequency band of the audio data.

32. The method claim 1, comprising sending a network message comprising an indication of the relative timing of the actuation and the interval.

33. An apparatus comprising a microphone for recording audio data, and a controller configured to determine the audio data to identify the actuation of an inhaler and breath of a user by performing the method of claim 1.

34-36. (canceled)

37. A device configured to perform the method of claim 1, wherein the device is configured to communicate with a server, wherein the communication comprises the transfer of data from the device to the server.

38. A system comprising a plurality of devices each configured to perform the method of claim 32, a server adapted to receive said network messages, and a controller configured to identify a group of said devices based on the network messages.

39. (canceled)
